(19) United States
(12) Patent Application Publication
(10) Pub. No.: US 2008/0161833 A1
(43) Pub. Date: Jul. 3, 2008

(54) JOINT PROSTHESIS

(76) Inventor: Charles D. Ray, Santa Barbara, CA (US)

Correspondence Address: DICKE, BILLIG & CZAJA, FIFTH STREET TOWERS, 100 SOUTH FIFTH STREET, SUITE 2250, MINNEAPOLIS, MN, 55402, US

(21) Appl. No.: 11/618,953

(22) Filed: Jan. 2, 2007

(51) Int. Cl. A61B 17/08; A61F 2/02

(52) U.S. Cl. 606/151; 623/11.11

(57) ABSTRACT

A joint prosthesis is described. The joint prosthesis includes a polymeric membrane defining opposing first and second ends and a graft surface opposite a bearing surface, and mesh backings attached to the membrane. In particular, a first mesh backing portion attached along the first end of the membrane on the graft surface, and a second mesh backing portion attached along the second end of the membrane on the graft surface. In this regard, the first and second mesh backing portions are configured for attachment to respective opposing bony surfaces of a body joint such that the membrane folds between the surfaces of the body joint and defines an articulation surface along the bearing surface of the membrane.

### FIELD OF THE INVENTION

The invention relates to prosthetic bearing surfaces for rotating or sliding joints of the human body and to the means of attachment of such prostheses to the underlying bone of the joints.

### BACKGROUND

As the population ages, the accumulative wear and tear on individual joints of the body progressively degrades and some may even become destroyed. This process is often painful and may thus limit or restrict comfortable or essential daily activities of the afflicted person. With progression, this painful disability may become so limiting as to shorten the person's life.

Patients now undergo joint replacements with prostheses but they too have a limited time span. Ordinary joints have two opposing surfaces that articulate or slide against each other. Joint motion is normally constrained by surrounding capsular ligaments. Joints are generally covered with a firm, wet, slippery cartilage that serves as a bearing surface. Further, each joint is surrounded by a sac or capsule that protects and further limits the joint, supplying it with its lubricating fluid, secreted by a contained gland. The resultant joint has very low friction, even under load.

However, joints can be forced to slide into abnormal positions, called subluxation. With degeneration, the joint may exhibit abnormal laxity or rigidity, become displaced chronically and the fluid source and capsule may dry and become inflamed with the associated degenerating joint becoming arthritic.

Artificial joints have shortcomings. The most common shortcomings are related to: (1) biocompatibility of the implant materials, (2) the attachment of the materials to the underlying bone bed, (3) the simple wearing out or fracturing of the articulating materials; and (4) the dispersal of the wear debris of the degraded material into neighboring tissues and lymph channels. Small body joints such as those of the ancillary facets of the spine, digits and the jaw are particularly lacking in adequate prostheses for replacement.

Normal joint bearing cartilage surfaces appear quite smooth and glistening to the naked eye but have numerous tiny variations (scoring) present in the contour that are closely matched with the other, opposing surfaces of the joint. Although highly polished prior to implantation, prosthetic joint surfaces have microscopic imperfections or peaks that wear away or are shed during use. After the opposing surfaces become worn in to each other the wear slows but never totally stops. If the opposing surfaces of a prosthesis are of differing hardness, the softer will be abraded (grooved or scratched) by the harder one. In addition, the softer material may smear onto the harder one and form an adherent film. Finally, long-term, progressive load bearing may lead to fatigue of the articulating surface material causing it to crack or have an accelerated wearing out. Particles of the abraded or cracked polymer may be transported into neighboring tissue or into the lymphatic channels. The byproducts of joint degradation may result in untoward changes in the tissues surrounding the prosthesis.

With the above Background in mind, improvements to, and advancement of, a prosthesis to replace degenerated or arthritic joints, particularly the many small ones as found on ancillary facet joints of the spine, digits and the jaw will be welcomed by surgeons and by patients having painful, enlarged degenerative joints.

### SUMMARY

One aspect of the present invention provides a joint prosthesis. The joint prosthesis includes a polymeric membrane defining opposing first and second ends and a graft surface opposite a bearing surface, and mesh backings attached to the membrane. In particular, a first mesh backing portion attached along the first end of the membrane on the graft surface, and a second mesh backing portion attached along the second end of the membrane on the graft surface. In this regard, the first and second mesh backing portions are configured for attachment to respective opposing bony surfaces of a body joint such that the membrane folds between the surfaces of the body joint and defines an articulation surface along the bearing surface of the membrane.

Another aspect of the present invention provides a method of replacing a portion of a joint located between opposing bony surfaces of a body. The method includes providing a joint prosthesis including a polymeric membrane defining opposing longitudinal ends and first and second mesh backing portions attached to a respective one of the opposing longitudinal ends on a graft surface of the membrane. The method additionally includes attaching the first mesh backing portion to a first bony surface of the joint, and attaching the second mesh backing portion to a second bony surface of the joint opposite the first bony surface. The method further includes folding an unbacked portion of the membrane between the opposing bony surfaces to define a contacting articulation surface.

Other aspects of the invention provide membrane defined by a folded thin sheet, strip or ribbon of a low friction tissue acceptable film, such as polytetrafluoroethylene (Teflon®) or similar polymer. When the membrane is folded onto itself, two opposing surfaces of the folded membrane form articulating surfaces. In one embodiment, the membrane does not adhere to body surfaces or to prepared bone, and the low friction film/membrane is permanently affixed to a thin, porous woven fabric. The porous fabric provides attachment locations/surfaces suitable for anchoring to bony surfaces of the joint. For this additional aspect of the prosthetic joint device, an open mesh backing made of woven tissue-acceptable polymer fibers such as Dacron®, high-molecular-weight polyethylene (HMWPE), cotton or suitable fabric is bonded to the back of the membrane. In this regard, the moderately loosely woven backing material will more completely attach to the prepared, supporting bony surfaces by tissue ingrowth.

The prepared film and backing provide a means to prevent the potential problem of film loosening. The portion at the folded free end of the film to which no bony attachment is desired, remains without such a fabric. Still further, the film and backing may have additional hygroscopic, hydrogel components added that promote wetting and tissue bonding of the backing fabric. In one embodiment, the articulating surfaces are hydrophobic which simultaneously promote improved capillary wetting, low-friction sliding as well as inhibition of tissue ingrowth.

Additionally means are disclosed that are useful in attaching the fabric-bonded fixed ends and sides of the novel joint composite to the bone, while leaving the free loop of film unattached. The center portions of the fold freely slide against each other, forming the articulating surfaces.

Another aspect of the present invention provides an appropriate surgical procedure to prepare the opposing bone beds for appropriate smooth support of the film and further reestablishes or closes the preexisting tissue capsule (bursa) around the prosthesis at the end of the procedure for particular joint replacement. In this regard, the invention and surgical procedure provide for attaching the prepared prosthesis to the prepared bone bed. The novel implant can be trimmed to fit during the surgical implantation procedure and therefore configured for application to a variety of joint replacements.

The final assembly of the prosthesis appropriately restores painless function to the diseased or degenerated joint by providing the surgeon with an adaptable prosthesis to properly fit the application. The articulating film may include a radio-opaque marking or visualizing substance or means such that the device surface conditions may be later evaluated using x-ray or scanner imaging visualization.

The shaped composite film with backing is positioned on and may be attached to the prepared bone surfaces using currently available metal or polymer bone staples.

Another embodiment of the novel device applies a sealing means, by radiofrequency coagulation or laser tissue bonding to the outer margins of the folded insert which then may contain a natural lubricant such as hyaluronic acid gel or a non-absorbable lubricant. Closing the joint capsule also retards or prevents additional tissue growth into the cavity formed by the folding.

### BRIEF DESCRIPTION OF THE DRAWINGS

Embodiments of the invention are better understood with reference to the following drawings. The elements of the drawings are not necessarily to scale relative to each other. Like reference numerals designate corresponding similar parts. The Figures diagrammatically illustrate a preferred embodiment of the present invention.

FIG. 1 illustrates a perspective view of a joint prosthesis according to one embodiment of the present invention.

FIG. 2 illustrates an oblique perspective magnified view of a joint prosthesis in position between bony joint components.

FIG. 3A illustrates a cross-sectional view of the joint prosthesis of FIG. 1 in a neutral or resting position.

FIG. 3B illustrates a cross-sectional view of the joint prosthesis of FIG. 1 in an upwards position.

FIG. 3C illustrates a cross-sectional view of the joint prosthesis of FIG. 1 in a downwards position.

### DETAILED DESCRIPTION

In the following detailed description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments of the present invention can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

Referring to the figures, like numerals indicate like parts throughout the several views.

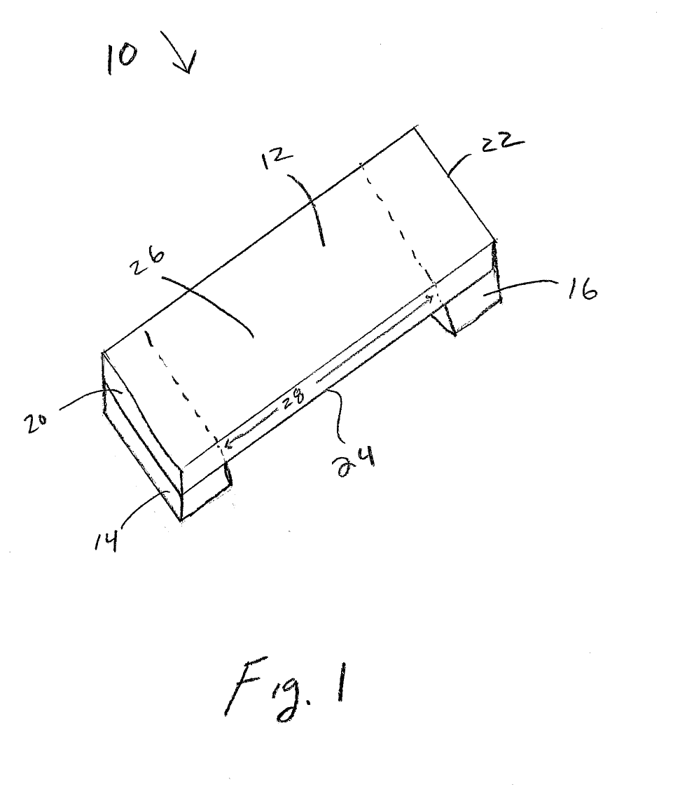

FIG. 1 schematically illustrates a perspective view of a joint prosthesis 10 according to one embodiment of the present invention. The joint prosthesis 10 includes a polymeric membrane 12, and first and second mesh backings 14, 16. As a point of reference, a thickness of the membrane 12 and backings 14, 16 is exaggerated in FIG. 1 for purposes of explanation. Along these same lines, and as made more clear below, the membrane 12 is highly flexible and thus the device 10 is in no way limited to the shape schematically represented in FIG. 1.

In one embodiment, the membrane 12 is made from a polymer such as polytetrafluoroethylene, fluorinated ethylene propylene copolymer, perfluoroalkoxy, copolymers of ethylene and tetrafluoroethylene, ethylene, silicone, polyester, or nylon. Regardless, the membrane 12 is thin and highly flexible. The membrane 12 defines opposing first and second ends 20, 22 and a graft surface 24 opposite a bearing surface 26. The mesh backings 14, 16 are attached to the graft surface 24. In one embodiment, the mesh backings 14, 16, when attached to the membrane 12, do not extend along an entire length of the membrane 12, but rather leave an exposed portion 28 along the membrane 12. In particular, the first mesh backing 14 is attached at or adjacent the first end 20 of the membrane 12 on the graft surface 24, and the second mesh backing 16 is attached at or adjacent the second end 22. By preferably sizing the mesh backings 14, 16 as shown, the membrane 12 can readily fold along the exposed portion 28 as described below. To this end, and in one embodiment, the first and second mesh backing 14, 16 are configured for attachment to respective opposing bony surfaces of a body joint such that the membrane 12 folds between the surfaces of the body joint and defines an articulation surface along the bearing surface 26.

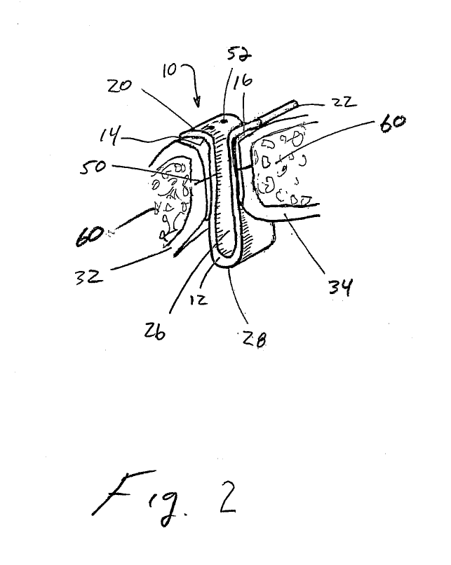

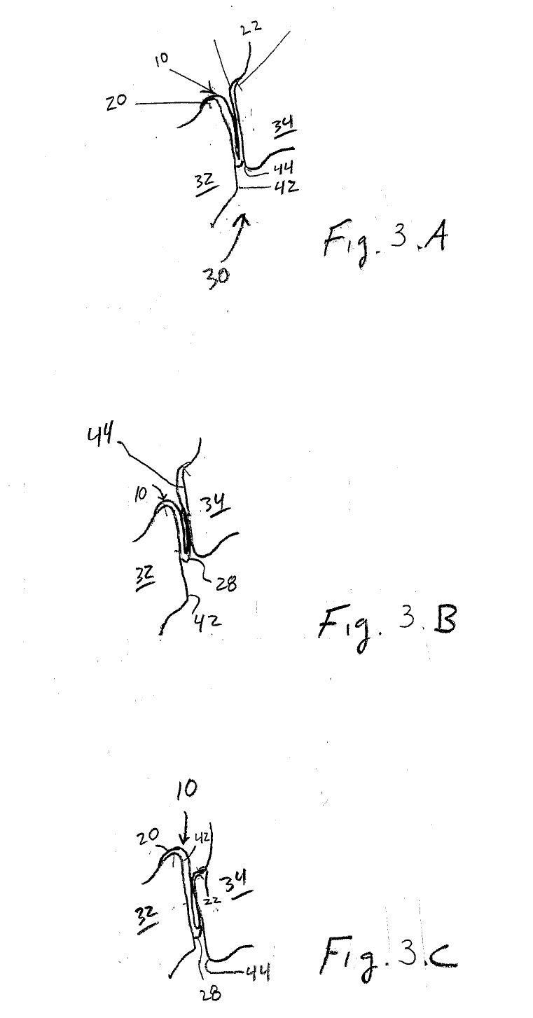

During use, and in general terms, the prosthesis 10 is implanted between two bony structures of a human joint, with the membrane 12 being folded upon itself in a manner allowing for multiple articulating positions. With this in mind, FIG. 2 shows the prosthesis 10 of FIG. 1 where a diagrammatic illustration of the joint is in its initial neutral position. The bonded and attached ends of the membrane 12 and the lower free folded portion 28 are illustrated. In one embodiment, anchoring pins 50 are attached at a midpoint of bones 32, 34. In one embodiment, upper attachment pins 52, such as staples, for example, are indicated. In this illustration the membranous periosteum 60 of the bones 32, 34 are also diagrammed. FIGS. 3A-3C illustrate the joint prosthesis 10 in three articulating positions: FIG. 3A neutral, FIG. 3B raised; and FIG. 3C lowered.

FIG. 3A illustrates a side view of the joint prosthesis 10 in a neutral or resting position relative to a body joint 30. The body joint 30 is defined by the first bone 32 and the second bone 34. The first bone 32 defines a first bony surface 42, and the second bone 34 defines a second bony surface 44. In one embodiment, the mesh backing 14 is attached to the first bone 32, for example, and the mesh backing 16 is attached to the second bone 34. Attachment of the device 10 to the bones 32, 34 can be accomplished by employing suitable attachment means, such as pins, screws, staples, adhesives, and the like.

As best illustrated in FIG. 3B, in one embodiment the unbacked portion 28 of the membrane 12 folds between the bones 32, 34 such that the bearing surface 26 is folded onto itself to define an articulation interface between the bony surfaces 42, 44. In one embodiment, the membrane 12 is a fluorinated membrane having low static and dynamic friction coefficients to promote movement along the articulation interface.

In one embodiment, the unbacked portion 28 defines a looped free end between the bones 32, 34, and the looped free folded end is without backing from the attachment points at edges of the backings 14, 16, and beyond. In one embodiment, small suitable staples or other attachment means anchor the fixed, fabric backed ends 20, 22 to the prepared bony surfaces 42, 44. The attaching means or pins also show the relative positions of the movable, articulating joint halves relative to each other. This also illustrates that the folded unbacked portion 28 moves up and down effectively, in one embodiment, one half the distance of the motion of the opposing surfaces of the joint.

Surgical opening and closure of the original joint capsule are not illustrated but are well known to surgeons. An additional polymeric or tissue grafted capsule, also not shown, may be attached to surround the novel prosthetic body joint.

In this embodiment the upper, fabric bonded portions of the composite film or membrane are attached to the bone using metal or polymer staples. Tissue ingrowth with additional fixation occurs after a few weeks normally and enhancing biochemical factors can accelerate this. During normal excursions, the prostheses, just as with all normal joints will exhibit limitations in motion largely determined by the fibrous or ligamentous capsule of the joint. Limitations in joint motion or expansion of the novel joint lining film will continue rely on joint fibers and surrounding tissues. The bonded and folded portions of the film or membrane are length adjustable during implantation to account for the preferred range of motion, preventing later dislocation or avulsion of attachment to the underlying bone.

By way of the above novel device and attendant surgical procedure, a low friction joint motion is achieved effectively providing normal joint action. The thicker the film the greater coverage of irregularities on the underlying bone, however the prepared joint space and laxity of its capsule limit the space availability and thus the thickness of the bonded and free portions of the film.

For one skilled in the art, other membrane composites and means of attachment to bone may be substituted without changing the intent and performance of the invention.

### Method and Example of Use

With reference to FIGS. 1-3C, the membrane 12 and the fabric backings 14, 16 are shown. In FIG. 1 the relative positions of the joint are shown in a neutral, elevated and lowered position to illustrate the attachments and free motion portions of the novel body joint. The surgeon exposes the diseased or painfully arthritic joint using a standard surgical approach. He then opens the joint capsule, removes the diseased cartilage and by cutting and abrasion, resurfaces the joint. The preferred final shapes of the two opposing joint surfaces are established in this way and a suitable portion of the novel joint membrane is trimmed to fit. The extent of joint mobility aids in determining the length and size of the membrane in its fabric bonded and non-bonded portions. A trial fitting is performed and when suitable the fabric bonded ends are suitably affixed to the prepared bone surfaces using suitable means such as biotolerant metal (such as titanium) or polymer pins or staples of a permanent or biodegradable form (such as polylactic acid or polygalactone) are driven through the bony cortex into the underlying cancellous or spongy bone. The range of motion of the new joint surfaces is determined by displacement using surgical grasping instruments.

When a suitable configuration and action have been achieved, the original joint capsule is closed or a suitable substitute (tissue graft or polymer) then surrounds the revised joint. A suitable lubricant such as hyaluronic acid may be injected into the closed capsule. If possible a normally lubricating suitable synovial fluid-secreting gland may be transplanted onto the capsule. The tissues are all closed, terminating the procedure.

### Advantages

The device has the novel ability, through the use of a simple technique, to provide a low-friction, flexible, weight-bearing surface that primarily replaces the cartilage and ragged bony surfaces of degenerative joints of the body. The attachment to bone is provided only at the outer margins of the joint and the more complex attachment into the amputated ends of long bones used in total joint replacements is eliminated. The residual joint, even if damaged by arthritis can be surgically reshaped to accommodate the novel prosthesis without the need to make major revisions to the bone or remnant cartilage of the joint.

Examples of diseased joint that may benefit from the present novel prosthesis include those joints have principally bidirectional flexion-extension motions (called diathrodial joints), such as the knees, fingers and toe joints, the elbows, facet joints of the vertebral column and the tempromandibular joints. The compound rotary motion of hip and shoulder joints would require different, more complex shapes of the present prosthesis.

The novel device described herein addresses issues in prosthetic joint design, namely, low friction surfaces required for proper joint function, reduced wear between the opposing surfaces with reduced creation of particles of wear and simplified, improved attachment of the device to the supporting bones of the joint. Further, the membrane or film of the prosthesis when attached to the supporting bone resists dislocation from that attached position.

Although specific embodiments have been illustrated and described herein, it will be appreciated by those of ordinary skill in the art that a variety of alternate and/or equivalent implementations may be substituted for the specific embodiments shown and described without departing from the scope of the present invention. This Specification is intended to cover any adaptations or variations of the specific embodiments discussed herein. Therefore, it is intended that this invention be limited only by the claims and the equivalents thereof.

While certain embodiments of the invention have been described, it should be understood that various changes, adaptations and modifications may be made therein by those skilled in the art without departing from the spirit and scope of the invention.

The application of this novel prosthetic body joint is devoid of the undesirable side effects associated with total joint replacements particularly for small joints such as listed above. The appropriate polymeric film or membrane, backing and means of attachment will not result in rejection phenomena or erosion of the underlying bone. There will be a minimum of wear of the sliding surfaces of the prosthesis and there should be no release of abraded portions as seen with cast or machined polymers used in joint replacements. Further, since the film is continuously of the same material there will be no reaction among dissimilar materials often seen in prostheses made of unlike substances, such as metal-to-metal or metal-to-polymer joint replacements. The preferred effects of the novel prosthesis are immediate and continuous.

The prosthesis in a preferred embodiment is disclosed here although persons skilled in the mechanical and polymer arts can adapt the concept to a variety of means to cause desirable low-friction bearing surfaces. No other prosthetic body joint device or method serving this low friction application of a bonded polymer film with attachment to a prepared, degenerated sliding body joint, adjustable by the surgeon at time of implantation is known to exist at this time.

What is claimed is:

1. A joint prosthesis comprising:
a polymeric membrane defining opposing first and second ends and a graft surface opposite a bearing surface;
a first mesh backing portion attached along the first end of the membrane on the graft surface; and
a second mesh backing portion attached along the second end of the membrane on the graft surface;
wherein the first and second mesh backing portions are configured for attachment to respective opposing bony surfaces of a body joint such that the membrane folds between the surfaces of the body joint and defines an articulation interface along the bearing surface of the membrane.

2. The joint prosthesis of claim 1, wherein the polymeric membrane is a fluorinated polymeric membrane.

3. The joint prosthesis of claim 1, wherein the polymeric membrane is selected from the group consisting of polytetrafluoroethylene, fluorinated ethylene propylene copolymer, perfluoroalkoxy, copolymers of ethylene and tetrafluoroethylene, ethylene, silicone, polyester, and nylon membranes.

4. The joint prosthesis of claim 1, wherein at least one of the first and second mesh backing portions includes a woven fiber backing.

5. The joint prosthesis of claim 1, wherein at least one of the first and second mesh backing portions includes a woven polyester fiber backing.

6. The joint prosthesis of claim 1, wherein one of the membrane and the mesh backing portions includes a radio opaque marker.

7. The joint prosthesis of claim 1, wherein an unbacked portion of the membrane extends between the first and second mesh backing portions.

8. The joint prosthesis of claim 7, wherein the membrane defines a length between the first and second ends, and further wherein the unbacked portion of the membrane is at least 50% of the length.

9. A method of replacing a portion of a joint located between opposing bony surfaces of a body, the method comprising:
providing a joint prosthesis including a polymeric membrane defining opposing longitudinal ends and first and second mesh backing portions attached to a respective one of the opposing longitudinal ends on a graft surface of the membrane;
attaching the first mesh backing portion to a first bony surface of the joint;
attaching the second mesh backing portion to a second bony surface of the joint opposite the first bony surface; and
folding an unbacked portion of the membrane between the opposing bony surfaces to define a contacting articulation surface.

10. The method of claim 9, wherein the polymeric backing includes one of an additive and a coating configured to inhibit tissue growth into the membrane.

11. The method of claim 9, wherein attaching the first and second mesh backing portions to a respective bony surface of the joint includes adding a tissue bonding additive to at least one of the mesh backing portions and the bony surfaces.

12. The method of claim 9, wherein attaching the first and second mesh backing portions to a respective bony surface of the joint includes stapling each of the mesh backing portions to a respective one of the bony surfaces.

13. The method of claim 9, wherein folding an unbacked portion of the membrane includes sealing outer margins of the membrane to define a joint capsule.

14. The method of claim 13, wherein sealing includes laser tissue bonding.

15. The method of claim 13, wherein sealing includes radiofrequency coagulation.

16. The method of claim 13, further comprising:
adding a lubricant to the capsule.

17. The method of claim 13, further comprising:
transplanting a fluid-secreting gland into the capsule.

18. The method of claim 9, wherein the joint is a diathrodial joint.

19. The method of claim 9, wherein the joint is a vertebral joint.
